(19) United States
(12) Patent Application Publication
(10) Pub. No.: US 2013/0211260 A1
(43) Pub. Date: Aug. 15, 2013

(54) Apparatus and method for minimally invasive length measurement within a hollow organ

(71) Applicant: Thomas KÖTHE, Leutenbach (DE)

(72) Inventor: Thomas KÖTHE, Leutenbach (DE)

(21) Appl. No.: 13/585,862

(22) Filed: Aug. 15, 2012

(30) Foreign Application Priority Data
Aug. 25, 2011 (DE) 102011081546.5

(51) Int. Cl. A61B 8/12; A61B 1/00; A61B 8/00

(52) U.S. Cl. CPC A61B 8/12; A61B 8/461; A61B 8/44; A61B 1/0008
USPC 600/449

(57) ABSTRACT

An apparatus for minimally invasive length measurement within a hollow organ is provided. The apparatus has an endoscopic instrument, an ultrasound head having an ultrasound transmitter and ultrasound receiver, an ultrasound reflector, an electrical connection, a control and display device and a mechanical connection. The ultrasound reflector is arranged at one end of the endoscopic instrument. The ultrasound head is arranged at one end of the mechanical connection extending into a channel of the endoscopic instrument and is guided herein. The ultrasound head is aligned with the ultrasound reflector and the distance between the two can be changed. The ultrasound head and the control and display device are connected by the electrical connection to exchange electrical signals. The control and display device generates an ultrasound wave and detects the wave reflected at the ultrasound reflector using the ultrasound receiver and determines the delay time of the wave.

### CROSS REFERENCE TO RELATED APPLICATIONS

This application claims priority of German application No. 10 2011 081 546.5 filed Aug. 25, 2011, which is incorporated by reference herein in its entirety.

### FIELD OF INVENTION

The present application relates to an apparatus for minimally invasive length measurement within a hollow organ. Furthermore, the present application relates to a method for minimally invasive length measurement within a hollow organ.

### BACKGROUND OF INVENTION

In the field of application of minimally invasive, interventional radiology and surgery, therapeutic interventions are performed for instance, with the aid of diagnostic, radiological image methods, such as for instance angiography, cardiology or neuroradiology, in vascular, e.g. peripheral arterial occlusive diseases, or in a biliary system, e.g. a tumorous occlusion of the biliary system in order to reestablish or stabilize the quality of life of a patient. There is the need here within cardiology, neuroradiology or angiography to identify the pathological change in the vascular system, in the form for instance of stenoses, perforations or aneurysms by these diagnostic image methods and to eliminate the same using suitable means such as dilatation of the stenosis, implantation of a stent, clipping or coiling. Crucial to the success of such an intervention is often a precise determination of ranges or distances in the vascular system. By way of example, the accuracy of fit of a surgical implant to be applied, e.g. a stent or a balloon, is of huge importance since if the size of the implant and the size of the area to be closed and/or extended do not correspond, serious complications may result. A critical point is often the branching of a vessel, since with incorrectly implanted stents or imprecise balloon dilatation one of the two branches is closed which may indicate an insufficient supply of the affected tissue. This has large-scale consequences on the myocardial muscle or in the even more critical brain area and may even lead to a heart attack or a stroke. In less existential cases, an examiner must remove a stent prior to the final dilatation or reintroduce the same in the body in order to open this occlusion. This not only places an additional burden on the patient's health, but also results in extra costs for staff and equipment.

To ensure that the treating physician chooses the correct surgical implant to apply, such as a stent or a balloon, a precise determination of the dimensions of an affected region or region of interest (ROI) is essential in order to prevent residual stenoses and/or additionally open aneurysms.

Length data based on parameters of recorded images and the device positions associated therewith were previously calculated by different analysis software. The algorithms of this software are based on mathematical models, which are cited in specialist literature and used in medical research. It is absolutely necessary in this way to subject the system to extensive adjustments prior to use of the software. The responsibility for any inaccuracies of any type, which result from the use of the existing software, lies in the hands of the user. Manufacturers often indicate that the precision of the adjustment has a direct influence on the precision of the calculated variables, such as for instance artery diameter or ejection fraction. Large deviations from expected values generally produce an incorrect adjustment. In order to ensure accuracy of the adjustment, reference measurements are regularly needed. Different analysis models and their necessary image data input are also often not compatible and are a frequent source of error.

### SUMMARY OF INVENTION

The object of the present application consists in specifying an apparatus which enables an accurate length measurement within a hollow organ. The object of the application is further to describe a method for minimally invasive length measurement within a hollow organ.

The application achieves this object with an apparatus for minimally invasive length measurement having the features of the first independent claim and a method for minimally invasive length measurement having the features of the second independent claim.

A basic idea behind the application is an apparatus for minimally invasive length measurement within a hollow organ. It includes an endoscopic instrument, an ultrasound head, having an ultrasound transmitter and an ultrasound receiver, an ultrasound reflector, an electrical connection means, a control and display means and a mechanical connection means. The ultrasound reflector or the ultrasound head is arranged at one end of the endoscopic instrument. The ultrasound head or the ultrasound reflector is arranged at one end of the mechanical connection means. The mechanical connection means extends at least partially into a channel of the endoscopic instrument and is guided herein. The distance of the ultrasound head relative to the ultrasound reflector can be changed and the ultrasound head can be aligned with the ultrasound reflector. The electrical connection means can be connected to the ultrasound head and the control and display means, in order to exchange electrical signals, and the control and display means is designed to generate an ultrasound wave by the ultrasound transmitter, to detect ultrasound waves reflected at the ultrasound reflector with the ultrasound receiver and to determine the delay time of the wave.

An endoscopic instrument is understood to mean a tube or rod-type device having a length of approx. 0.3 to 1.5 m and a diameter of approx. 1 to 20 mm, which can be used for minimally invasive examinations or interventions. Conventional endoscopic instruments in most cases have working channels, into which micromechanical devices, such as small forceps or grippers, can be inserted and with which examining or intervening processes can be performed in an examination region. A hollow organ may be a vessel, such as a blood vessel or a lymph vessel, a transport tube, e.g. for saliva or an organ, such as for instance a small intestine, a stomach or an airway or feed pipe, in a human or animal. Ultrasound transmitters, ultrasound receivers and ultrasound reflectors are components or functional units, which are commercially available in numerous embodiments and also in the smallest of installation sizes. The combination of ultrasound transmitter and ultrasound receiver is frequently offered in a component, i.e. in a housing, as an ultrasound head. An electrical connection means can be understood to mean metallic wires or a total of electrical cables. The mechanical connection means may for instance be embodied as a flexible plastic rod with a round cross-section. By guiding the mechanical connection means in a channel of the endoscopic instrument, the distance of the ultrasound head relative to the ultrasound reflector can be changed for instance such that the length of the mechanical connection means, which protrudes beyond the one end of the endoscopic instrument, is varied. The control and display means may be a computer or an electronic computer having a monitor or a display for instance, which is embodied to transmit control signals to the ultrasound transmitter and the ultrasound receiver and to receive therefrom electrical signals. The control and display means is also designed to execute mathematical computing operations. The control and display means is not necessarily an independent device, the functions can also be executed by a device, which primarily processes other tasks, e.g. the computing unit of an imaging facility, like an x-ray device.

With the disclosed apparatus, the ultrasound reflector may be arranged at one end of the endoscopic instrument and the ultrasound head may be arranged at one end of the mechanical connection means. In another embodiment, the ultrasound head is arranged at the one end of the endoscopic instrument and the ultrasound reflector is arranged at the one end of the mechanical connection means. This arrangement features in that the electrical connection means does not have to be guided by way of the mechanical connection means, since the reflector, which is assumed as such in a passive embodiment, does not have to be connected to the electrical connection means.

The most basic function which can be implemented by the disclosed apparatus is that by using an ultrasound head and a defined reflector, which are attached to a catheter in a mechanically decoupled manner, and/or are arranged thereon, the distance between the head and reflector can be precisely determined through the delay time of the echo.

The ultrasound head or the ultrasound reflector is fixedly connected to the one end of the mechanical connection means and the ultrasound reflector or ultrasound head is fixedly connected to the end of the endoscopic instrument. The ultrasound head is aligned with the ultrasound reflector. The mechanical connection means has rigidity, so that the ultrasound head and the ultrasound reflector are aligned relative to one another at least up to a predeterminable distance.

The function of the disclosed apparatus is that the ultrasound transmitter, the ultrasound receiver and the ultrasound reflector are aligned with one another, i.e. that the ultrasound wave transmitted by the ultrasound transmitter strikes the ultrasound reflector in a straight line, said ultrasound reflector reflecting the wave and the reflected wave then striking the ultrasound receiver in a straight line. The covered range of the ultrasound wave is twice the length of the range to be measured. This property is achieved in that the ultrasound head or the ultrasound receiver is fixedly connected to the mechanical connection means at one end of the mechanical connection means. The ultrasound reflector or the ultrasound head is fixedly connected to the endoscopic instrument at one end of the endoscopic instrument. The mechanical connection means has a rigidity, so that the ultrasound head and the ultrasound reflector are aligned relative with one another at least up to a predeterminable distance. The rigidity of the mechanical connection means, which is embodied for instance as a plastic rod, ensures the alignment. The greater the rigidity here, the better the alignment of the ultrasound head and the ultrasound reflector can be ensured. In practice, an ideally rigid endoscopic instrument extending over a range of a few millimeters or centimeters may nevertheless be unwanted if for instance the endoscopic instrument has to be guided through a tube-type vessel having small radii. The rigidity of the mechanical connection means is expedient so that the ultrasound head and the ultrasound reflector are aligned with one another at least up to a predeterminable distance. The distance may be specified for instance as a multiple, e.g. twice the diameter of the hollow organ or for instance ten times the diameter of a tube-type vessel. The rigidity and the geometric properties of the mechanical connection means, such as for instance cross-section, can be determined for instance with the aid of known mechanical laws, from the mechanical properties of the material of the mechanical connection means such as the elasticity module, the transverse forces to be expected, such as flows within the vessel, and the tolerances in respect of alignment accuracy, e.g. an allowed tolerance range of reflected waves striking the ultrasound sensor having a diameter of one millimeter.

An electronic means which is arranged within the endoscopic instrument or on an endoscopic instrument is designed to execute at least one function, for which the control and display means is designed.

In this embodiment, an electronic means assumes at least one function of the control and display means. In this case this may be a display for instance, which is attached to the endoscopic instrument, and assumes the function of the display of the result of the length measurement.

A further basic idea behind the application relates to a method for minimally invasive length measurement within a hollow organ, which includes the following method steps:
S1) positioning an ultrasound head with a transmitter and a receiver at a starting point of the range to be measured within the hollow organ and aligning the transmitter and the receiver with an end point of the range to be measured within the hollow organ, wherein no significant material inhomogeneity exists in the straight-line connection between the starting point and end point;
S2) positioning an ultrasound reflector at the end point of the range to be measured and aligning the ultrasound reflector with the ultrasound head;
S3) determining the sound speed of ultrasound in the medium which lies in the straight-line connection between the starting point and the end point;
S4) emitting a wave through the ultrasound head and measuring the delay time of the wave from the ultrasound head to the ultrasound reflector and back to the ultrasound head;
S5) calculating the range between the starting point and end point, wherein the delay time of the wave from the ultrasound head to the ultrasound reflector and back to the ultrasound head and the sound speed are included in the calculation.

In the first method step, the ultrasound head, which includes a transmitter and a receiver, is positioned at the starting point of the range to be measured within the hollow organ and the transmitter and the receiver are aligned with an end point of the range to be measured within the hollow organ, wherein no significant material inhomogeneity exists in the straight-line connection between the starting point and the end point. Alignment here is understood to mean that when emitting ultrasound waves, the end point lies in the direction of the emitted ultrasound waves. The request that no significant material inhomogeneity is permitted to exist in the straight-line connection between the starting point and the end point ensures that there is no vascular wall or body's own object between the starting point and end point, e.g. an interventional aid, which hampers the length measurement and would render the same unusable.

In the next method step, the ultrasound reflector is positioned at the end point of the range to be measured and the ultrasound reflector is aligned at the ultrasound head.

In the third method step, the sound speed of ultrasound is determined in the medium which lies in the straight-line connection between the starting point and the end point. This may take place for instance by calibration in the specialist literature or by a measurement.

In the next method step, the ultrasound head sends an ultrasound wave. The delay time of the wave from the ultrasound head to the ultrasound reflector and back to the ultrasound head is measured.

In the last method step, the range between the starting point and the end point is calculated. In this way the calculation includes the delay time of the wave from the ultrasound head to the ultrasound reflector and back to the ultrasound head, and the sound speed of the ultrasound in the medium, which exists in the straight-line connection between the starting point and the end point. The simplest calculation formula reads:

s=0.5*v*t,

with the sought length s, the sound speed of ultrasound in the medium v and the measured delay time of the wave t.

Also conceivable is a method for minimally invasive length measurement within a hollow organ, wherein the ultrasound head with the transmitter and the receiver is positioned at the starting point of the range to be measured within the hollow organ and the transmitter and the receiver are aligned with the end point of the range to be measured within the hollow organ, wherein no significant material inhomogeneity exists in the straight-line connection between the starting point and end point, and wherein the ultrasound reflector is positioned at the end point of the range to be measured and the ultrasound reflector is aligned with the ultrasound head, which includes the described methods steps S3 to S5.

The method uses a disclosed apparatus, as was described previously.

After the fourth method step, the result of the calculation of the distance between the starting point and end point is expediently output on the control and display means or on the electronic means. The control and display means and the electronic means were described beforehand in conjunction with the disclosed apparatus.

The ultrasound head and/or the ultrasound reflector is/are positioned using at least one image, which is obtained by an imaging method. In this process the at least one image includes the starting point and/or the end point. The imaging method, e.g. using an x-ray device, generates an image, a series of images or a live image, in which the starting and the end point of the range length to be measured are identified and the positioning of the ultrasound head and the ultrasound reflector are easy to implement.

### BRIEF DESCRIPTION OF THE DRAWINGS

The embodiments illustrated in more detail below represent embodiments of the present application. Further developments result from the subsequent figures and description, in which:

FIG. 1 shows a tube-type vessel with a stenosis;

FIG. 2 shows an apparatus for minimally invasive length measurement within a hollow organ;

FIG. 3 shows a flow chart of a method for minimally invasive length measurement within a hollow organ;

FIG. 4 shows components of an apparatus for minimally invasive length measurement within a hollow organ in a tube-type vessel.

### DETAILED DESCRIPTION OF INVENTION

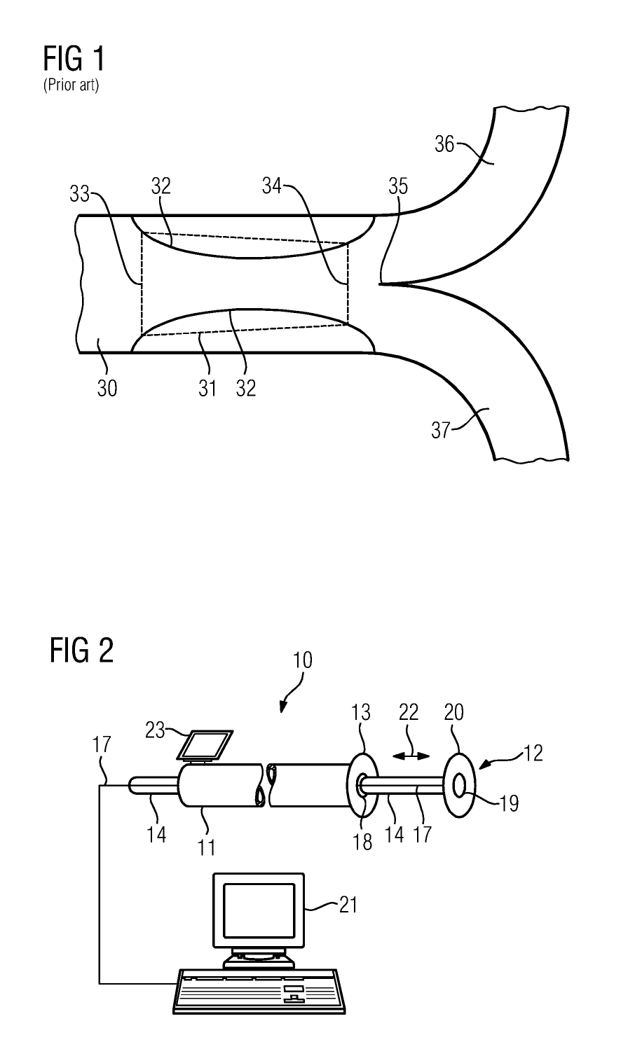

FIG. 1 shows a schematic representation of a tube-type vessel 30 having a stenosis 32. The vessel 30 branches into a first branch 36 and into a second branch 37 at point 35. In order to select a suitable stent for widening the stenosis 32, the area or region of interest 31 (ROI) must be measured as accurately as possible. The length, i.e. the length of the range from the straight line 33 to the straight line 34, of the stenosis is determined precisely. If the size of the stent is selected incorrectly, it may close one of the two vessel branches 36 or 37 for instance, as a result of which the supply of this branch is negatively affected.

FIG. 2 shows a schematic representation of a disclosed apparatus 10 for minimally invasive length measurement within a hollow organ. It includes an endoscopic instrument 11, an ultrasound head 12, having an ultrasound transmitter 19 and an ultrasound receiver 20, an ultrasound reflector 13, an electrical connection means 17, a control and display means 21, an electronic means 23 and a mechanical connection means 14. The ultrasound reflector 13 is arranged at one end of the endoscopic instrument 11 and is fixedly connected thereto. The ultrasound head 12 is arranged at one end of the mechanical connection means 14 and is fixedly connected hereto. The mechanical connection means 14 extends in this embodiment completely into a channel 18 of the endoscopic instrument 11 and is guided herein. The distance of the ultrasound head 12 to the ultrasound reflector 13, is, as indicated by the double arrow 22, changeable. This can be achieved for instance by sliding the mechanical connection means 14 in or out at the end of the mechanical connection means 14 shown to the left in FIG. 2. The mechanical connection means 14 has a rigidity, so that the ultrasound head 12 and the ultrasound reflector 13 are aligned with one another at least up to a predeterminable distance. The electrical connection means 17 can be connected to the ultrasound head 12 and the control and display means 21, in order to exchange electrical signals. The control and display means 21 is designed to generate an ultrasound wave by the ultrasound transmitter 19, to detect the ultrasound wave reflected at the ultrasound reflector 13 using the ultrasound receiver 20 and to determine the delay time of the wave. In this embodiment, the electrical means 23 is a display, which is attached to the endoscopic instrument 11 and indicates the result of the length measurement for instance.

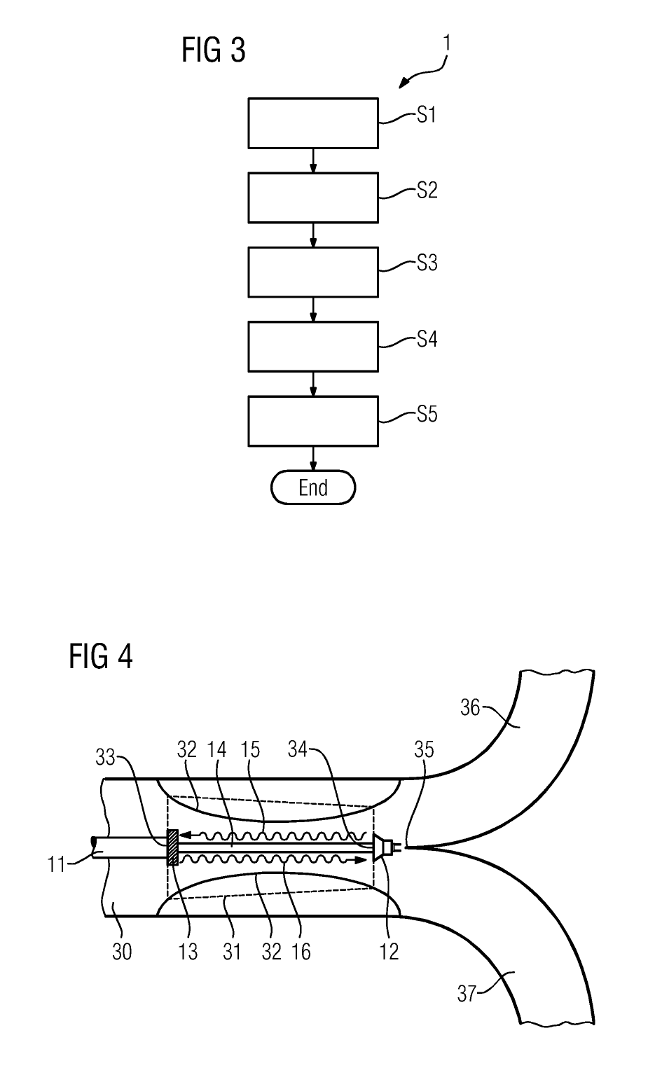

FIG. 3 shows a flow chart of a disclosed method 1. The following methods steps can be identified:
S1) positioning an ultrasound head having a transmitter and a receiver on a starting point of the range to be measured within the hollow organ and aligning the transmitter and the receiver with an end point of the range to be measured within the hollow organ, wherein no significant material homogeneity exists in the straight-line connection between the starting point and the end point;
S2) positioning an ultrasound reflector at the end point of the range to be measured and aligning the ultrasound reflector with the ultrasound head;
S3) determining the sound speed of ultrasound in the medium, which exists in the straight-line connection between the starting point and the end point;
S4) emitting a wave through the ultrasound head and measuring the delay time of the wave from the ultrasound head to the ultrasound reflector and back to the ultrasound head;
S5) calculating the range between the starting point and end point, wherein the calculation includes the delay time of the wave from the ultrasound head to the ultrasound reflector and back to the ultrasound head, and the sound speed.

FIG. 4 finally shows a schematic representation of an embodiment of components of a disclosed apparatus for minimally invasive length measurement within a hollow organ 30. The representation ties in with the representation in FIG. 1 and likewise shows a schematic representation of the tube-type vessel 30 with the stenosis 32. The vessel 30 branches out into a first branch 36 and a second branch 37 at point 35. A suitable stent must be selected in order not to risk closure of one of the two vessel branches 36 and 37. To this end, the examination region 31 and its length, i.e. the length of the range from the straight line 33 to the straight line 34, is to be determined precisely. To this end, a disclosed apparatus is used for minimally invasive length measurement within a hollow organ. It includes an endoscopic instrument 11, an ultrasound head 12, with an ultrasound transmitter and ultrasound receiver (not shown), an ultrasound reflector 13, a mechanical connection means 14 and an electrical connection means (also not shown) and a control and display means. The ultrasound reflector 13 is arranged at one end of the endoscopic instrument 11 and is fixedly connected hereto. The ultrasound head 12 is arranged at one end of the mechanical connection means 14 and is fixedly connected hereto. An imaging method (not shown), e.g. an x-ray device, generates a live image, which includes the examination region 31 with the starting and end point of the range length to be measured. The ultrasound head 12 is now positioned on the starting point 34. The mechanical connection means 14 extends into a channel of the endoscopic instrument 11 and is guided herein. The distance of the ultrasound head 12 from the ultrasound reflector 13 can be changed by changing the length of the mechanical connection means 14 between the ultrasound head 12 relative to the ultrasound reflector 13. The ultrasound reflector is then positioned at the end point of the distance to be measured. The mechanical connection means 14 has a rigidity so that the ultrasound head 12 and the ultrasound reflector 13 are aligned with one another at least up to a predeterminable distance. An active alignment of the ultrasound reflector 13 with the ultrasound head 12 is not necessary. In the next step, the sound speed of ultrasound in the medium which lies in the straight-line connection between the starting point and the end point is determined. This medium is what is found inside the hollow organ and which surrounds the ultrasound head 12, the mechanical connection means 14 and the ultrasound reflector 13. The sound speed of ultrasound can be determined by measuring or by looking up specialist literature. After actuation of a button by an operating person for instance, the control and display means (not shown), initiates the emission of an ultrasound wave 15 through the ultrasound transmitter (not shown) in the ultrasound head 12. Since the ultrasound head 12 is aligned with the ultrasound reflector 13, the ultrasound wave 15 strikes the ultrasound reflector 13 and is reflected as an ultrasound wave 16. The reflected ultrasound wave 16 strikes the ultrasound receiver (not shown) in the ultrasound head 12, since the ultrasound reflector 13 is aligned with the ultrasound head 12. The control and display means (not shown) registers the arrival of the reflected ultrasound wave 16 at the ultrasound head and determines the delay time of the wave from the ultrasound head 12 to the ultrasound reflector 13 and back to the ultrasound head 12, i.e. the signal delay time from the point of transmission to the point of reception. The control and display means (not shown) finally calculates the length of the range between the starting point 34 and end point 33, wherein the calculation includes the delay time of the wave from the ultrasound head 12 to the ultrasound reflector 13 and back to the ultrasound head 12, and the sound speed, and is output on a monitor. The functions of the control and display means (not shown) can also be detailed in whole or in part by the device (not shown) for the imaging method.

In summary, it may be said that the features of the disclosed apparatus and the disclosed method compared with conventional software length analyses is that the risk of a misinterpretation of the conditions of the human organism is prevented and life-threatening complications during the medical intervention are thus minimized. A qualified additional treatment can also take place with the aid of the application and various risks, such as residual stenosis or incompletely covered aneurisms, which may result in an acute heart attack or even in a stroke, are minimized.

A significant savings potential also forms the basis of the application, since it is highly probable that the examiner can grip the correct “tool” catheter or balloon simultaneously, and the use of further instruments is not needed. This reduces the use of expensive instruments and the duration of the actual treatment is also shortened by the rapid and reliable analysis of the region to be examined.

1. An apparatus for minimally invasive length measurement within a hollow organ, comprising:
an endoscopic instrument;
an ultrasound head comprising an ultrasound transmitter and ultrasound receiver;
an ultrasound reflector;
an electrical connection;
a control and display device; and
a mechanical connection,
wherein the ultrasound reflector or the ultrasound head is arranged at one end of the endoscopic instrument or at one end of the mechanical connection,
wherein the mechanical connection extends at least partially into a channel of the endoscopic instrument and is guided herein,
wherein distance of the ultrasound head relative to the ultrasound reflector can be changed,
wherein the ultrasound head is aligned with the ultrasound reflector,
wherein the electrical connection connects the ultrasound head and the control and display device to exchange electrical signals, and
wherein the control and display device is designed to generate an ultrasound wave by the ultrasound transmitter, to detect the ultrasound waves reflected at the ultrasound reflector using the ultrasound receiver, and to determine a delay time of the ultrasound wave.

2. The apparatus as claimed in claim 1, wherein the ultrasound head or the ultrasound reflector is fixedly connected to the one end of the mechanical connection or is fixedly connected to the end of the endoscopic instrument, and wherein the mechanical connection is rigidity so that the ultrasound head and the ultrasound reflector are aligned with one another at least up to a predeterminable distance.

3. The apparatus as claimed in claim 1, wherein an electronic device is arranged within or on the endoscopic instrument and is designed to execute at least one function of the control and display device.

4. A method for minimally invasive length measurement within a hollow organ, comprising:
positioning an ultrasound head having a transmitter and a receiver on a starting point of a range to be measured within the hollow organ;
aligning the transmitter and the receiver with an end point of the range to be measured within the hollow organ, wherein no material homogeneity exists in a straight-line connection between the starting point and the end point;
positioning an ultrasound reflector at the end point of the range to be measured;
aligning the ultrasound reflector with the ultrasound head;
determining a sound speed of ultrasound in a medium in the straight-line connection between the starting point and end point;
emitting an ultrasound wave through the ultrasound head;
measuring a delay time of the ultrasound wave from the ultrasound head to the ultrasound reflector and back to the ultrasound head; and
calculating the range between the starting point and the end point from the sound speed and the delay time.

5. The method as claimed in claim 4, wherein the method is executed by an apparatus as claimed in claim 1.

6. The method as claimed in claim 4, wherein the calculated range between the starting point and the end point is output on a control and display device or on an electronic device.

7. The method as claimed in claim 4, wherein the ultrasound head and/or the ultrasound reflector is positioned using at least one image comprising the starting point and/or the end point.
